(19) United States
(12) Patent Application Publication
(10) Pub. No.: US 2025/0236917 A1
(43) Pub. Date: Jul. 24, 2025

(54) RAPID AND ACCURATE SINGLE-NUCLEOTIDE POLYMORPHISM DETECTION BY FLUOROPHORE-NUCLEIC ACID INTERACTION

(71) Applicant: SNPsnipe, Inc., Germantown, MD (US)

(72) Inventors: Hai Yan, Bethesda, MD (US); Rui Yang, Germantown, MD (US)

(73) Assignee: SNPsnipe, Inc., Germantown, MD (US)

(21) Appl. No.: 19/033,939

(22) Filed: Jan. 22, 2025

Related U.S. Application Data
(60) Provisional application No. 63/623,527, filed on Jan. 22, 2024.

(51) Int. Cl. C12Q 1/6886; C12Q 1/6816; C12Q 1/6827

(52) U.S. Cl. CPC C12Q 1/6886; C12Q 1/6816; C12Q 1/6827

(57) ABSTRACT

The present disclosure relates generally to devices and methods for rapidly and accurately diagnosing single-nucleotide polymorphisms. According to one or more embodiments, a system for identifying a single-nucleotide polymorphism (SNP) includes an oligonucleotide primer with a SNP recognition sequence, a fluorophore conjugated to the oligonucleotide primer at a conjugation site to form a fluorophore-conjugated oligonucleotide primer, a polymerase enzyme to extend the oligonucleotide primer upon binding to an oligonucleotide strand with a complimentary sequence to the oligonucleotide primer, forming a double stranded deoxyribonucleic acid (DNA) amplicon, and wherein the fluorophore-conjugated oligonucleotide primer is a sole primer in the system, such that the fluorophore only emits a fluorescent signal when the fluorophore-conjugated oligonucleotide primer binds to a sequence specifically complementary to the SNP recognition sequence.

### CROSS-REFERENCE TO RELATED APPLICATION

This application claims the benefit of U.S. Provisional Patent Application No. 63/623,527, filed Jan. 22, 2024, which is incorporated by reference herein in its entirety.

### BACKGROUND

Embodiments described herein relate generally to systems, devices, and methods for rapidly and accurately diagnosing single-nucleotide polymorphisms (SNPs). More specifically, embodiments relate to systems, devices, and methods comprising a deoxyribonucleic acid (DNA) extension and amplification platform with fluorophore-nucleic acid interaction-based nucleic acid sequence recognition. The present disclosure relates, in other embodiments, to systems, devices, and methods comprising fluorophore-nucleic acid interactions and linear extensions for single nucleotide polymorphism (SNP) detection.

There are many challenges in the field of molecular diagnosis due to the complexities inherent in the existing diagnostic platforms, which results in confusion and inefficiencies. The current conventional technologies are associated with ambiguity, including issues such as false positives, false negatives, high costs, and challenges in data interpretation. For example, polymerase chain reaction (PCR) and next generation sequencing (NGS) are two major platforms that have their own inherent challenges. Overcoming these obstacles is of paramount importance for advancing patient care and facilitating medical research in the area of SNP identification and patient diagnosis and treatment.

The completion of the human genome sequence and the emergence of genome SNP databases has ushered in a transformative era in medicine, shifting from traditional approaches to precision medicine. This modern paradigm relies on the growing understanding of SNPs and their correlations with a wide range of clinical phenotypes, including conditions such as but not limited to infectious diseases, cancers, asthma, neuropsychiatric disorders, sickle cell disease, and drug metabolism.

SNPs play a pivotal role in disease prevention and treatment strategies. SNP variants involve subtle changes at the genetic level, typically single base pair differences in the DNA sequence. These changes can be difficult to detect compared to the presence or absence of an entire pathogen.

Fundamental requirements for advancing precision medicine are the abilities to test patients swiftly and accurately for SNPs and other genetic mutations. In support of these requirements, the United States (US) Food and Drug Administration (FDA) has mandated that the pharmaceutical industry publicly disclose SNP data assessed during the drug licensing process.

Various SNP detection and analysis methods have been devised, each based on distinct principles. Traditional methods for SNP detection often face challenges related to non-specific amplification. Sanger sequencing, another mutation detection approach, is also time-consuming, costly, and, most critically, constrained by its limited sensitivity (with a detection limit of approximately 20% mutation allele frequency). Common SNP detection methods are also often constrained by contextual sequence dependencies, enzyme efficiency, or fidelity, and necessitate professionally trained technicians, sophisticated equipment, comprehensive sample preparation, and meticulous result interpretation.

The present disclosure is directed to systems, devices, and methods for accurately and efficiently identifying or assessing the presence of SNPs in a given sample.

### BRIEF SUMMARY

Systems and methods for identifying or assessing gene alterations including single-nucleotide polymorphism (SNP) are described. Importantly, systems and methods that can detect SNPs with accuracy, specificity, and speed in a cost-effective manner are described.

In some embodiments, a system for detecting SNPs utilizes specific primers labeled with fluorophores. The SNP specific primers labeled with fluorophores can amplify signals only when they precisely match the target DNA sequence after primer extension. Such accuracy ensures specificity and precision in detecting genetic variations. The systems can mitigate the issue of false positives by employing the SNP specific primers for linear extension during loop-mediated amplification, instead of the conventional cycling method used in PCR. Such approaches ensure that signals only originate from the mutant DNA itself, excluding any contributions from PCR artifacts. In embodiments, the systems can achieve robust amplification because of the cycling primers of loop-mediated isothermal amplification (LAMP). The systems can achieve seamless linear extension, facilitated by the fluorophore-labeled SNP specific LAMP loop primers, all within a single reaction.

According to one or more embodiments, a system for identifying a single-nucleotide polymorphism (SNP) comprises an oligonucleotide primer comprising a SNP recognition sequence; a fluorophore conjugated to the oligonucleotide primer at a conjugation site to form a fluorophore-conjugated oligonucleotide primer; a polymerase enzyme to extend the oligonucleotide primer upon binding to an oligonucleotide strand with a complimentary sequence to the oligonucleotide primer, forming a double stranded deoxyribonucleic acid (DNA) amplicon; and wherein the fluorophore-conjugated oligonucleotide primer is a sole primer in the system, such that the fluorophore only emits a fluorescent signal when the fluorophore-conjugated oligonucleotide primer binds to a sequence specifically complementary to the SNP recognition sequence.

In some embodiments, the system further comprises an amplification platform.

In other embodiments, the amplification platform is a loop-mediated isothermal amplification (LAMP) platform.

Yet, in other embodiments, the fluorophore is in a quenched state when the fluorophore-conjugated oligonucleotide primer does not undergo extension.

In one or more embodiments, the fluorophore is in a quenched state when a complementary strand formed in the double stranded DNA amplicon contains a mismatch at the conjugation site.

In other embodiments, the fluorophore is in a quenched state when a complementary strand formed in the double stranded DNA amplicon contains a mismatch within two or more bases of the conjugation site.

In some embodiments, the fluorophore-conjugated oligonucleotide primer is a loop primer.

Yet, in other embodiments, the fluorophore is conjugated to a guanine (dG), adenine (dA), cytosine (dC), or thymine (dT).

In some embodiments, the fluorophore is conjugated to an oligonucleotide base that is about 1 to about 5 nucleotides from the 3′ end of the oligonucleotide primer.

In some embodiments, the system does not include a reverse primer.

According to one or more embodiments, a method of treating a patient comprises obtaining a sample from the patient, the sample comprising an oligonucleotide; lysing and preparing the sample for an assay to identify a single-nucleotide polymorphism (SNP); combining a fluorophore-conjugated oligonucleotide primer and a polymerase enzyme with the oligonucleotide, a fluorophore conjugated to the fluorophore-conjugated oligonucleotide primer at a conjugation site and the fluorophore-conjugated oligonucleotide primer comprising a SNP recognition sequence; determining whether the oligonucleotide in the sample comprises the SNP based on whether the fluorophore emits a fluorescent signal, the fluorophore-conjugated oligonucleotide primer being a sole primer in the method, such that the fluorophore only emits the fluorescent signal when the fluorophore-conjugated oligonucleotide primer binds to a sequence specifically complementary to the SNP recognition sequence; and treating the patient if the SNP is detected in the sample.

In some embodiments, determining whether the oligonucleotide in the sample comprises determining if the fluorophore remains quenched, indicating a DNA duplex mismatch.

In other embodiments, treating the patient includes performing a molecular diagnosis for a condition or a disease, wherein the condition or the disease is, optionally, tuberculosis (TB), COVID-19, meningitis, encephalitis, congenital infections, sepsis, an acute coronary syndrome, a histocompatibility issue, an adverse drug reaction, pre-eclampsia, cancer, or any combination thereof.

Yet, in other embodiments, the method further comprises employing a digital platform, wherein optionally, the digital platform is for applications in circulating cell free DNA, circulating cell free RNA, circulating tumor DNA (ctDNA), circulating tumor RNA (ctRNA), circulating pathogen, minimal residual disease (MRD), single-cell analysis, and/or spatial diagnostics.

According to one or more embodiments, a method for identifying a single-nucleotide polymorphism (SNP) in an oligonucleotide sample comprises combining a fluorophore-conjugated oligonucleotide primer comprising a SNP recognition sequence, a polymerase enzyme, and the oligonucleotide sample, a fluorophore conjugated to the fluorophore-conjugated oligonucleotide primer at a conjugation site; determining whether the oligonucleotide sample comprises the SNP based on whether the fluorophore emits a fluorescent signal; and confirming that the oligonucleotide sample includes the SNP when the fluorescent signal is emitted; wherein the fluorophore-conjugated oligonucleotide primer is a sole primer in the method, such that the fluorophore only emits the fluorescent signal when the fluorophore-conjugated oligonucleotide primer binds to a sequence specifically complementary to the SNP recognition sequence.

In some embodiments, the fluorophore-conjugated oligonucleotide primer is a loop primer.

In other embodiments, the fluorophore-conjugated oligonucleotide primer further includes one or more Locked Nucleic Acids (LNAs).

In other embodiments, the method does not include using a reverse primer.

### BRIEF DESCRIPTION OF VIEWS OF THE DRAWINGS

The following description accompanies the drawing(s), all given by way of non-limiting examples that may be useful to understand how the described method and composition may be embodied.

FIG. 1A is an illustration of an SNP-detecting primer design according to embodiments;

FIG. 1B is an illustration of a detection principle employed in systems for detecting gene alterations (SNPs), wherein the fluorophore becomes fluorescent in absence of a mismatch between primer and its annealing site on sample DNA template according to embodiments;

FIG. 1C is an illustration of a detection principle employed in systems for detecting gene alterations (SNPs), wherein the fluorophore remains quenched when there is a mismatch between primer and its annealing site on sample DNA template according to embodiments;

FIG. 2 is an illustration of a fluorophore-conjugated oligonucleotide primer during the loop-mediated isothermal amplification (LAMP) process according to embodiments;

FIG. 3 is an illustration demonstrating challenges in SNP detection mechanisms with traditional PCR processes;

FIG. 4 is an illustration depicting an amplification phase (initiation phase not shown) of a loop-mediated isothermal amplification (LAMP) process, demonstrating that SNP detection can be realized using a LAMP process according to some embodiments; and

FIG. 5 is an illustration of a method of identifying or assessing genetic alterations such as an SNP in a sample according to some embodiments.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present disclosure is defined solely by the claims. The features illustrated or described in connection with one exemplary embodiment may be combined with the features of other embodiments. Such modifications and variations are intended to be included within the scope of the present disclosure. Accordingly, aspects and features of every embodiment may not be described with respect to each embodiment, but those aspects and features are applicable to the various embodiments unless statements or understandings are to the contrary.

As used herein, the term “patient” or “user” refers to any subject, including but not limited to, plants, pathogens, animals (e.g., pets, farm animals, etc.), and humans. The patient may have a condition and/or disease or suspected of having a condition and/or disease and as such is being treated with a drug. In some instances, the patient is a mammal, such as a human, a premature neonate, neonate, infant, juvenile, adolescent, or adult thereof. In some instances, the term “patient,” as used herein, refers to a human (e.g., a man, a woman, or a child). In some instances, the term “patient,” as used herein, refers to plants, pathogen, or laboratory animal of an animal model study. The patient or subject may be of any age, sex, or combination thereof.

The term “treating” refers to administering a therapy in an amount, manner, or mode effective (e.g., a therapeutic effect) to improve a condition, symptom, disorder, or parameter associated with a disorder, or a likelihood thereof.

The terms “essentially” or “substantially” as used herein mean to a great or significant extent, but not completely.

The term “about” as used herein refers to any values, including both integers and fractional components that are within a variation of up to plus or minus 10% of the value modified by the term “about.” In some embodiments, “about” means plus or minus 1%, 2%, 3%, 4%, 5%, 6%, 7%, 8%, 9%, or 10% of the stated value.

Two notable limitations afflict current mainstream single-nucleotide polymorphism (SNP) detection methods, including hybridization-based methods and amplification-refractory mutation system (ARMS) methods. Hybridization-based methods, including taqman probes and molecular beacons, utilize the principle of complementary base pairing to identify specific alleles. DNA probes with known sequences are designed to bind to complementary sequences in the target DNA, and the strength of hybridization depends on the degree of sequence match, enabling SNP detection.

On the other hand, ARMS methods are PCR-based methods that use specially designed primers to selectively amplify DNA fragments containing a specific SNP allele, allowing for discrimination between different genotypes based on amplification success or failure. Allele-specific primers are used with a single nucleotide mismatch at the 3′ end, which only efficiently amplifies DNA fragments containing the matching allele due to the reduced DNA polymerase enzyme processibility caused by the mismatch. PCR products are visualized on an agarose gel, where the presence or absence of an amplified band indicates the genotype at the SNP locus.

SNPs often have minimal impact on hybridization in G-C-rich regions due to the strong bonding between guanine (G) and cytosine (C) bases. G-C pairs form three hydrogen bonds, compared to the two hydrogen bonds formed by adenine (A) and thymine (T) pairs. High G-C content leads to the formation of stable secondary structures, such as hairpins and self-dimers, which can hinder primer annealing and cause poor amplification efficiency, making it difficult to reliably detect SNPs in these areas. G-C rich regions are therefore more stable and less susceptible to disruptions caused by a SNP change. Thus, SNP recognition in G-C-rich regions proves considerably more challenging, as a SNP has minimal impact on hybridization.

As such, one limitation of these SNP detection methods is that designing assays for G-C-rich regions remains a significant challenge compared to A-T-rich regions because it is substantially easier to design primers, probes, or DNA clamps for less stable A-T-rich regions. Further, enhanced specificity in A-T-rich regions is possible especially when enhanced specificity is achievable through specialized nucleotide modifications such as lock nucleic acid (LNAs) or peptide nucleic acid (PNAs). LNAs and PNAs are specialized nucleotide modifications that significantly enhance the binding affinity of synthetic nucleic acid strands to their complementary DNA or RNA targets, primarily by altering the backbone structure to provide increased stability and specificity compared to natural nucleic acids. LNAs achieve this by “locking” the sugar ring conformation with a bridge between the 2′ and 4′ carbons, while PNAs replace the phosphodiester backbone with a pseudopeptide backbone, resulting in a neutral charge that improves binding capabilities.

Another limitation of the current strategies is their inherent susceptibility to errors, which causes them to rely on probability rather than absolute certainty. For example, in conventional hybridization-based methods, probes preferentially bind to mutant amplicons, but the probes will also incorrectly bind to wild-type amplicons and generate false-positive signals.

Similarly, in ARMS methods, mismatches in mutant-specific primers preferentially block amplification from wild-type templates, while permitting amplification from mutant templates. However, the blockage of wild-type templates is never perfect, and therefore, false-positive signals may be generated when wild-type templates are amplified. Consequently, regardless of the chosen mechanism, the background wild-type allele may be amplified, leading to false-positive signals. The interpretation of results relies on carefully setting cutoff values, which are easily influenced by various factors, such as differences in PCR efficiency introduced by variations in DNA quality and quantity between actual samples and control samples.

Therefore, there is a need for a faster and more accurate systems and methods for diagnosing gene alterations such as SNPs to support timely medical decisions and treatments.

Embodiments described herein overcome the foregoing challenges and are directed to directed to primers, systems, and methods for identifying, diagnosing and treating a patient with a SNP rapidly and accurately. Embodiments are directed to primers, systems, and methods comprising the genetic and biochemical assays for the detection of single-nucleotide polymorphism.

Some embodiments are directed to primers, systems, and methods comprising an isothermal PCR platform that combines the speed of loop-mediated isothermal amplification platform (LAMP) with the precision of fluorophore-nucleic acid interaction-based nucleic acid sequence recognition. Other embodiments are directed to primers, systems, and methods that perform rapid, multiplexable, portable, highly sensitive, and highly accurate detection of genetic alterations in relevant samples. Embodiments are directed to primers, systems, and methods that perform assays that provide a fluorescence readout in a binary fashion (“on” or “off”) and that can be completed very rapidly, e.g., in less than 30 minutes in some embodiments. In other embodiments, the assays can provide a fluorescence readout in a binary fashion and that can be completed in less than 10 minutes.

In some embodiments, the primers, systems, and methods are implemented to enable the identification of SNPs at low levels across various tissue types and qualities, in both DNA and RNA. For example, in some embodiments, the primers, systems, and methods are implemented to enable the identification of single mutations when they occur in only 1% or more of the DNA or RNA. In some embodiments, the primers, systems, and methods can accommodate impurities, enhancing versatility for field samples.

Embodiments are directed to a sequence-specific detection system comprising non-naturally occurring interactions between fluorophores and nucleic acids. The system includes a mechanism to conjugate one or more fluorophores to a nucleic acid base, or a thymine (dT), a guanine (dG), an adenine (dA), or a cytosine (dC), in a primer that includes a SNP recognition sequence.

In one or more embodiments, the systems and methods include a SNP detection primer with a fluorophore conjugated to a thymine (dT), a guanine (dG), an adenine (dA), or a cytosine (dC). In some embodiments, the systems and methods include a SNP detection primer with a fluorophore conjugated to a a thymine (dT), a guanine (dG), an adenine (dA), or a cytosine (dC) within about 1, about 2, about 3, about 4, or about 5 base pairs from the 3′ end of the primer, which terminates at the 3′ end with an inherent quencher, a cytosine (dC) or a guanine (dG). According to some embodiments, the primer includes a thymine (dT) within about 1, about 2, about 3, about 4, or about 5 base pairs from the 3′ end of an oligonucleotide primer, which terminates at the 3′ end with either a cytosine (dC) or guanine (dG), and the thymine (dT) is conjugated to the signaling fluorophore.

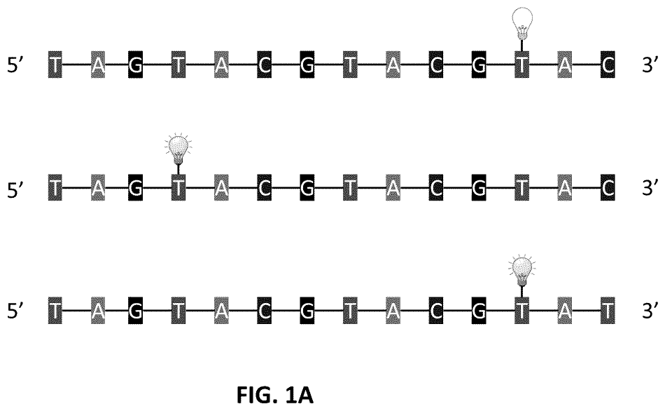

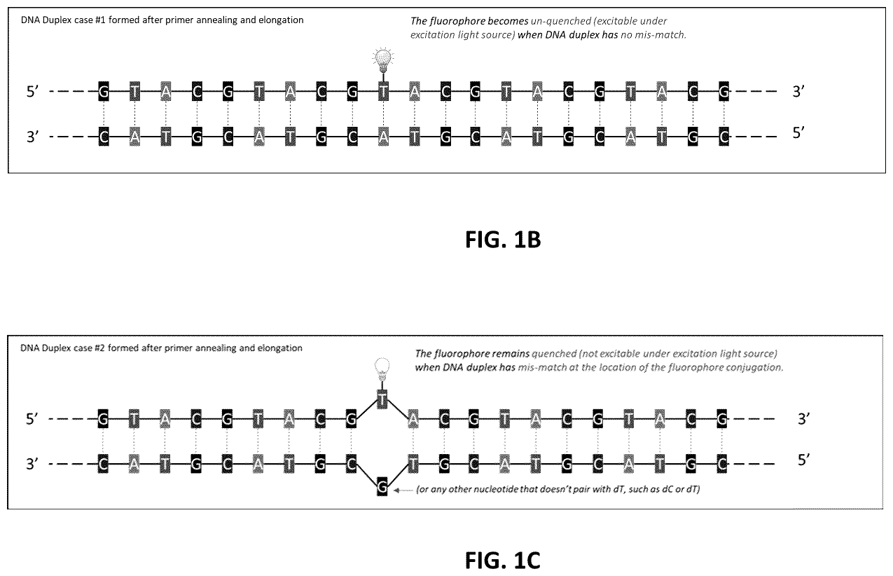

FIG. 1A illustrates a primer design according to one or more embodiments. In the first row, the desired primer is shown as an oligonucleotide with an illustrative SNP detection sequence and a fluorophore (light bulb) conjugated to a thymine (dT) positioned three bases away from the 3′ end. In this single stranded primer structure, the fluorophore remains quenched due to photoinduced electron transfer from the terminal 3′ cytosine (dC) positioned in proximity. While dC is illustrated, the 3′ terminal base can also be a guanine (dG). Each of guanine (dG) and cytosine (dC) are electron donors that readily transfer electrons, upon light excitation, to the fluorophore to quench the fluorophore's emission.

The middle row of FIG. 1A shows a comparative example of a primer that would not function as a suitable SNP detection primer as described herein. In this primer, the fluorophore is conjugated to a thymine (dT) position 11 bases away from the 3′ end, which is logistically too far away for the inherently electron transferring and quenching 3′ cytosine (dC) to effectively quench the fluorophore through photoinduced electron transfer.

In the third row, another comparative example of a primer that would not function as a suitable SNP detection primer as described herein includes a 3′ thymine (dT) at the terminal end. Although the fluorophore is conjugated to a thymine (dT) in close proximity to the 3′ end, like in row 1, where it could be quenched by photoinduced electron transfer from an inherent electron donating quencher, the 3′ terminal thymine (dT) is not a readily electron donating quencher.

In some embodiments, the system further includes a mechanism(s) to excite the fluorophores. The excitation of the fluorophores is dependent on the fluorophore-conjugated oligonucleotide serving as a primer and the complementary sequence of the annealing site being an exact match, e.g., dT pairing with adenine (dA), or dC pairing with dG. Upon an exact match pair and elongation by DNA polymerase, the fluorophore is excited and emits a longer wavelength light fluorescence, turning the fluorescent signal “on.” The detected fluorescent signal indicates an exact match pairing. When the fluorophore remains quenched, the signal remains off, indicating a mismatch.

In some embodiments, the systems and methods further include a fluorescence detector that converts the emitted fluorescent light signal into an electrical signal that is measured and analyzed. The fluorescent light emitted from the fluorophore enables real time monitoring of the amplification process as the amount of amplified DNA increases with each amplification cycle. In some embodiments, the amount of amplified DNA is quantified by measuring the fluorescent signal during each cycle to determine the cycle threshold value (Ct), which is inversely proportional to the DNA concentration. When the fluorophore becomes excited and turned on, by absorbing light at an absorption wavelength, the fluorophore emits light at a longer emission wavelength, which is detected by the fluorescence detector.

In one or more embodiments, the system further comprises a mechanism to detect a mismatch, e.g., dT pairing with dC or dG or dT, between the complementary sequence and the primer. If the system detects a DNA duplex mismatch (see FIG. 1C), due to a SNP, within a specific range of the fluorophore conjugation site, the fluorophore remains quenched, and the fluorescent signal is “off.” In some embodiments, the specific range is within 2 bases. In other embodiments, the specific range is within about 1 to about 5 bases, including any number of bases comprised therein. In other embodiments, the specific range can be more than 5 bases. In one or more embodiments, the specific range is about or any range between about 1, 2, 3, 4, 5, 6, 7, 8, 9, or 10 bases.

Some embodiments are directed to primers having an oligonucleotide strand comprising a thymine (dT) within about 1 to about 5 bases from a 3′ terminal end that terminates in a cytosine (dC) or a guanine (dG). In some embodiments, a fluorophore is conjugated to the thymine (dT) of the oligonucleotide strand to form a fluorophore-conjugated oligonucleotide primer.

In one or more embodiments, the fluorophore-conjugated primer can function as a loop primer in a LAMP platform.

In some embodiments, the oligonucleotide primer includes a fluorophore and an inherent electron transferring base, i.e., a guanine (dG) or a cytosine (dC) located at the primer's 3′ end, and the fluorophore remains quenched until annealed with a complementary strand and extended by a polymerase with exact base pair matching. All electrons in the matched DNA duplex have been paired perfectly, leaving no free electron available to quench the conjugated fluorophore, which will emit fluorescence in the presence of light source with excitable wavelength. However, when there is a mismatch within the proximity of the conjugated fluorophore, the un-paired electron(s) will continue to quench the fluorophore. While there may be cytosine (dC) and guanine (dG) bases in proximity to the fluorophore in the DNA duplex after elongation, internal cytosine (dC) and guanine (dG) bases will not quench the fluorophore. Only the 3′ terminal cytosine (dC) or guanine (dG) can quench the fluorophore in a single stranded oligonucleotide. In a double stranded DNA duplex, a fluorophore closer to the 5′ end will be turned on, regardless of the identify of the 3′ base (even if it is a cytosine (dC) or guanine (dG), illustrating the importance of the location of the fluorophore in the primer.

Described herein in embodiments are single stranded SNP recognition primers with a conjugated fluorophore within about five or less bases from the 3′ end, a guanine (dG) or cytosine (dC) at the 3′ end, and that satisfy two criteria. First, the primer remains quenched and is only excited/un-quenched upon forming the following described duplex. This second criteria is that the fluorophore is only unquenched/excited/emits fluorescence when it is annealed to a template oligonucleotide, elongated by DNA polymerase to form a duplex in which all base pairs are exactly matched to the primer sequence. Upon the following conditions, the fluorophore moves away from the 3′ end. The unquenched/excited/fluorescent fluorophore signal indicates that the complementary oligonucleotide includes the SNP sequence or a perfectly matched DNA duplex. The fluorophore remains quenched when a mismatch is present in the oligonucleotide either at the location of the fluorophore or within about 5 or less base pairs from the fluorophore, indicating that the oligonucleotide does not include the SNP sequence.

As shown in FIGS. 1B and 1C, in some embodiments, systems comprise a fluorophore-conjugate capable of recognizing a SNP. The fluorophore-conjugate is a fluorophore-conjugated oligonucleotide primer. In these embodiments, the fluorophore-conjugated oligonucleotide primer is terminated with either a dC or dG. In one or more embodiments, the primer includes a fluorophore modification is on a thymine (dT) positioned in close proximity to the 3′ end of the primer. In other embodiments, the primer includes a fluorophore modification on a dC, dG, dA, or dT.

When the fluorophore is linked closer to the 3′ end in the DNA duplex, the dG/dC pair at the 3′ end will quench the fluorophore. The fluorophore containing oligonucleotide therefore is a primer that becomes elongated to form a duplex and make the primer sequence (including the linked fluorophore) become the 5′ end of the duplex (as shown in FIGS. 1B and 1C). If the primer only anneals to a complementary template but is not elongated, the fluorophore will stay close to 3′ end, and the dG/dC pair will continue to quench it.

In some embodiments, fluorophore-conjugated oligonucleotide primers include a fluorophore attached to an oligonucleotide base, either directly or through a linker molecule. In one or more embodiments, the fluorophore is a fluorescein. In other embodiments, the fluorophore-conjugated oligonucleotide primer includes a different fluorophore, including but not limited to a fluorescein amidite, a carboxy-X-rhodamine, a hexachlorofluorescein, a tetrachloro fluorescein, an asymmetric xanthene dye (e.g., VIC phosphoramidite with fluorescence in the yellow-green part of the spectrum), a carboxytetramethylrhodamine (e.g., TAMRA), a cyanine3, a cyanine5, or any combination thereof.

In one or more embodiments, the fluorophore is directly attached to the oligonucleotide primer base. In other embodiments, the fluorophore is attached to the oligonucleotide primer base through a linker molecule.

In some embodiments, the fluorophore conjugation site is about 2 to about 6 bases from the 3′ end of the oligonucleotide primer. In other embodiments, the fluorophore conjugation site is about or in any range between about 2, 3, 4, 5, and 6 bases from the 3′ end of the oligonucleotide primer.

In one or more embodiments, the quencher of the fluorophore-conjugated oligonucleotide primer is an electron donor. In some embodiments, the quencher of the fluorophore-conjugated oligonucleotide primer is a guanine (dG) or a cytosine (dC).

In some embodiments, the fluorophore-conjugated oligonucleotide primer includes about 10 to about 30 oligonucleotides. In other embodiments, the fluorophore-conjugated oligonucleotide primer includes about 10 to about 100 oligonucleotides.

In one or more embodiments, the fluorophore-conjugated oligonucleotide primer further includes one or more Locked Nucleic Acids (LNAs). In other embodiments, the fluorophore-conjugated oligonucleotide primer further includes one or more non-natural nucleic acids.

The fluorophore-conjugated oligonucleotide primer includes an SNP recognition sequence with a sequence that recognizes a SNP in an oligonucleotide sample. In one or more embodiments, SNP detection is achieved by employing a primer with a conjugated fluorophore to a nucleotide base, which leverages a specific base pairing to turn on fluorescent signal.

In some embodiments, a loop-mediated isothermal amplification process or platform (LAMP) is used to leverage the fluorophore nucleotide interaction mechanism, as explained below. Compared to conventional methods that result in false positives, the fluorophore conjugated primer specific for the SNP is only involved in strand elongation but will not be used as a template for subsequent amplification.

LAMP is a DNA amplification technique used for diagnostics, operating at a constant temperature (i.e., about 60 to about 65° C.), unlike PCR that requires thermal cycling. LAMP employs four to six primers recognizing 6 to 8 distinct regions of target DNA for a highly specific amplification reaction. A strand-displacing DNA polymerase initiates synthesis, and two specially designed primers form “loop” structures to facilitate subsequent rounds of amplification through extension on the loops and additional annealing of primers. DNA products are ladder-like bands on electrophoresis gel, formed from various repeats of the short (e.g., 80-250 base pairs) target sequence, connected with single-stranded loop regions in long concatamers.

In other embodiments, other (non-LAMP) technological platforms can be used to leverage the fluorophore nucleotide interactions of a primer for SNP detection.

In one or more embodiments, the systems and methods include an isothermal amplification platform.

In one or more embodiments, the systems and methods include an amplification platform in which a polymerase enzyme elongates the oligonucleotide primer upon binding to an oligonucleotide strand with a complimentary sequence to the oligonucleotide primer, forming a double stranded deoxyribonucleic acid (DNA) amplicon, wherein the fluorophore-conjugated oligonucleotide primer is a sole primer in the system, such that the fluorophore only emits a fluorescent signal when the fluorophore-conjugated oligonucleotide primer binds to a sequence specifically complementary to the SNP recognition sequence.

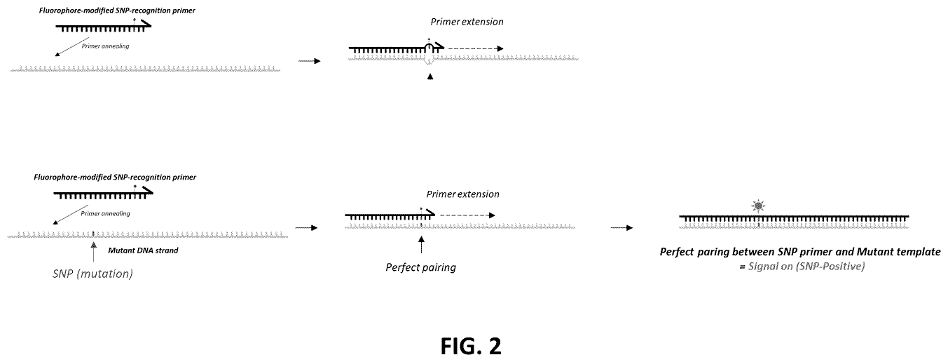

As illustrated in FIG. 2, systems and methods include a LAMP process in some embodiments. Such systems and methods include a fluorophore-modified SNP recognition primer. When the fluorophore-modified SNP recognition primer binds, combines, and anneals with a wild-type DNA strand that does not include the corresponding SNP, the primer will not form a base-pair match at the fluorophore-modified SNP site, forming a bump. The bump or DNA duplex mismatch between the SNP primer and the wild-type DNA strand results in the fluorophore remaining quenched, or not-excited. No signal or fluorescence means that the sample or wild type DNA strand does not include the SNP. However, when a mutant SNP containing DNA strand combines with and anneals to the SNP modified recognition primer, and perfect pairing occurs between the primer and the DNA strand, the fluorophore is excited, turning the signal or fluorescence on. The detected fluorescence signal means that the sample or mutant DNA strand does include the SNP.

In some embodiments, during the loop-mediated isothermal amplification (LAMP) process, the fluorophore on the fluorophore-conjugated oligonucleotide primer undergoes excitation based on two critical conditions. First, the fluorophore-conjugated oligonucleotide primer must undergo elongation or extension, resulting in the formation of a double-stranded DNA amplicon. Second, the complementary strand of the double stranded DNA amplicon must be an exact match to the DNA strand carrying the fluorophore modification.

In some embodiments, the conjugated fluorophore remains self-quenched or in a quenched state (and the fluorescent signal remains off) when the fluorophore-conjugated oligonucleotide primer remains as a single strand and doesn't undergo elongation or extension. In other embodiments, the conjugated fluorophore remains self-quenched when the fluorophore-conjugated oligonucleotide primer is elongated and forms a double-stranded DNA amplicon, but the complementary strand contains a DNA duplex mismatch at the site of the fluorophore conjugation. In other embodiments, the conjugated fluorophore remains self-quenched when the fluorophore-conjugated oligonucleotide primer is elongated and forms a double-stranded DNA amplicon, but the complementary strand contains a mismatch in close proximity, for example, within about 1 to about 5 base pairs, to the site of the fluorophore conjugation.

According to some embodiments, the methods and systems for SNP detection do not include a reverse primer. In other words, the fluorophore-conjugated primer only participates in strand elongation and is not used as a template for subsequent rounds of amplification. The fluorophore-conjugated oligonucleotide primer is the only primer in the system, such that the fluorophore only emits a fluorescent signal when the oligonucleotide primer binds to a sequence specifically complementary to the SNP recognition sequence in the primer.

In one or more embodiments, the fluorophore-conjugated primer only participates in the strand elongation but not the subsequent amplification (which would result in false positives).

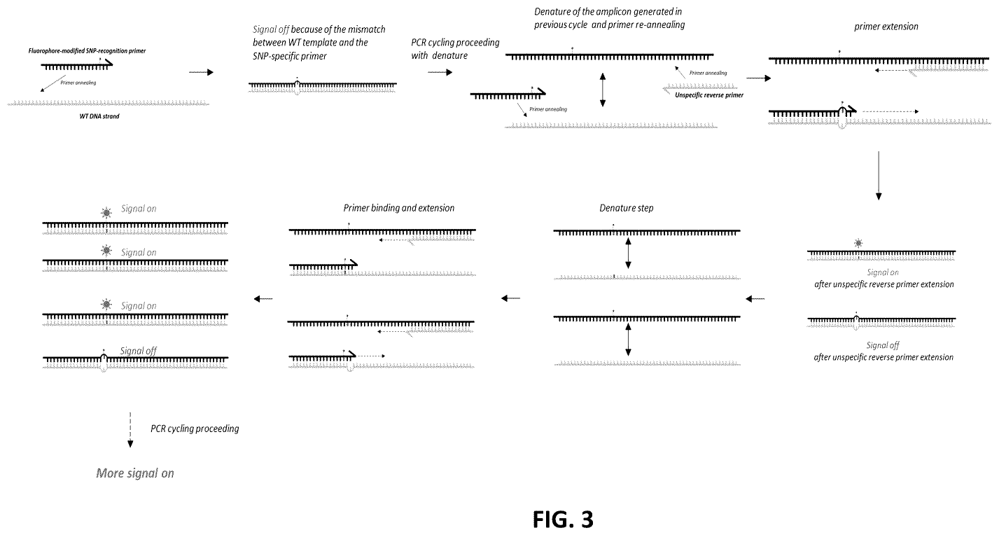

In contrast to embodiments described herein, which require a specific base pair match to turn on and off the signal, FIG. 3 that in traditional PCR, a signal will always be generated regardless of the template used. This occurs because after the fluorophore-conjugated oligonucleotide primer binds to the template and is elongated by the polymerase, the resulting double stranded DNA amplicon serves as a template for subsequent amplification. The reverse primer binds to the strand containing the fluorophore and uses it as a template to synthesize the complementary strand, ultimately producing a perfectly matched DNA duplex. At this point, the fluorophore becomes excitable since both of the aforementioned conditions are met. Therefore, a traditional PCR system can result in false positives, as the fluorescent signal is always turned on after amplification.

In some embodiments of the present disclosure, a multi-primer amplification system is used for SNP detection. Additional primers and competing primers can also be used to enhance speed and/or eliminate false positives.

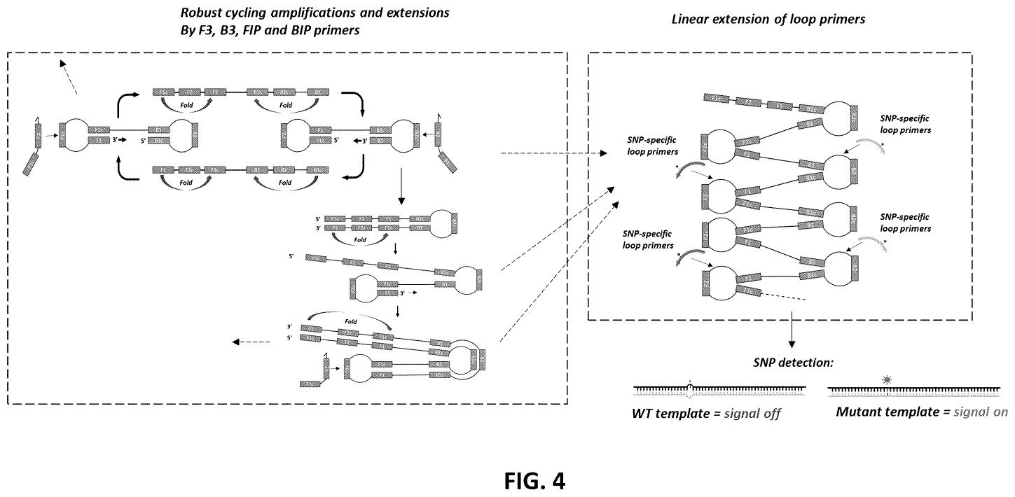

FIG. 4 illustrates a multi-primer amplification system based on a LAMP system according to embodiments. In some embodiments, a plurality of two or more fluorophore-labeled SNP specific loop primers are used for amplification and linear extension. In one or more embodiments, both robust amplification and seamless linear extension facilitated by the fluorophore-labeled SNP specific loop primers occur within a single reaction.

The resulting amplicons from the fluorophore-labeled SNP specific loop primers used in the LAMP system are not employed as templates for subsequent amplification. Cycling amplification is achieved using forward inner primers (FIP), backward inner primers (BIP), forward primers (F3), and/or backward primers (B3). The fluorophore-labeled SNP specific loop primers serve the dual purpose of expediting an initiation stage and facilitating the formation of a dumbbell structure, which serves as a starting point for robust cycling amplification and extension. Following this stage, the fluorophore-labeled SNP specific loop primer can also bind to a loop region and be elongated by the polymerase used in the LAMP system. However, the resulting amplicon will not be used as a template, effectively circumventing the issue encountered in traditional PCR systems. Therefore, unlike, a traditional PCR system, the multi-primer amplification system based on a LAMP system does not produce false positives. This approach ensures that signals only originate from the mutant DNA, SNP containing DNA itself, excluding any contributions from PCR artifacts. This approach eliminates false positives by utilizing the fluorophore-labeled SNP specific loop primers only once for linear extension during amplification, ensuring that non-specific amplification does not generate signals.

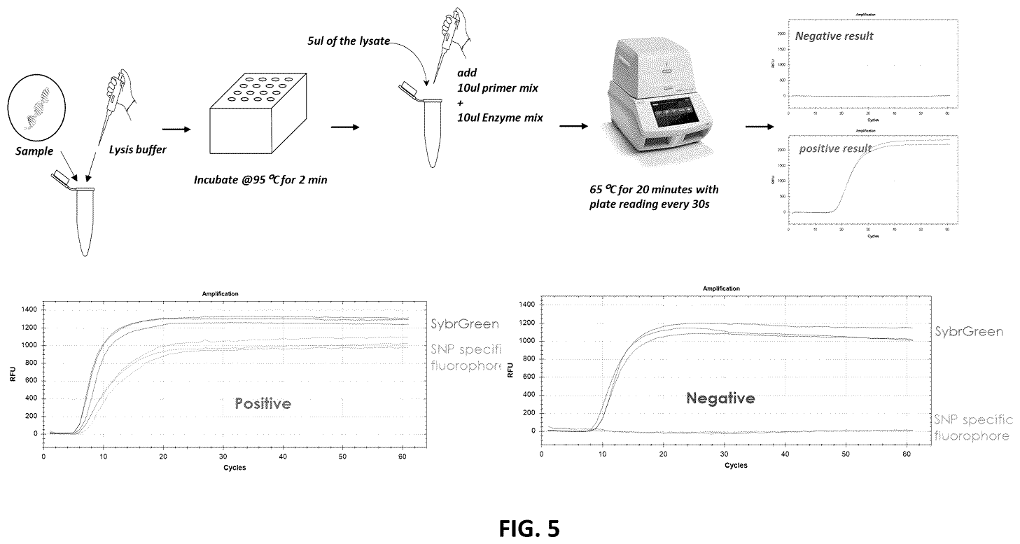

In some embodiments, the present disclosure is directed to a method of identifying or assessing SNP in a sample(s) of oligonucleotides. In some embodiments, as shown in FIG. 5, methods and systems include procuring a sample, lysing the sample, preparing the sample, and assessing the sample. In some embodiments, the samples include, but are not limited to, DNA, RNA, cell-free DNA (cfDNA), fresh tissues, frozen tissues, Formalin-Fixed Paraffin-Embedded (FFPE) tissues.

In one or more embodiments, systems and methods for identifying an SNP in a sample further include using a lysing enzyme to lyse the sample and using a polymerase to extend the primer.

In some embodiments, methods of identifying or assessing SNP further comprises treating a patient based on SNP detection. In one or more embodiments, the patient is treated for a condition or infectious disease, including but not limited to, tuberculosis (TB) (i.e., Mycobacterium tuberculosis (MTB)), COVID-19, the common cold, the flu (influenza), COVID-19, stomach flu (gastroenteritis), hepatitis, respiratory syncytial virus (RSV), meningitis, encephalitis, congenital infections, sepsis, an acute coronary syndrome, a histocompatibility issue, an adverse drug reaction(s), pre-eclampsia, or cancer, or any combination thereof.

In some embodiments, methods of identifying or assessing SNP are used as a surgical application for testing ambiguous specimens. In one or more embodiments, methods of identifying or assessing SNP are used as a surgical application for distinguishing tumor types. In some embodiments, methods of identifying or assessing SNP and structure variants are used as a surgical application for accurate margin assessment. In other embodiments, methods of identifying or assessing SNP or structure variants are used as a surgical application for confirming molecular features intraoperatively. Yet, in some embodiments, methods of identifying or assessing SNP are used to enable intraoperative clinical trials.

In one or more embodiments, methods of identifying or assessing SNP are used for diagnosing and treating infections, including but not limited to, upper respiratory infections, latrogenic infections, angioinvasion infections, sexually transmitted infections, food pathogen related infections, or any combination thereof.

In some embodiments, methods of identifying or SNP are used for liquid biopsy applications, for cfDNA, including SNPs, structure variants, copy number analysis, expression level profiling, methylation measurements, or any combination thereof.

In one or more embodiments, methods of identifying or assessing SNP and structure variants include implementing any digital platform. In other embodiments, the digital platform is for applications that include, but are not limited to, applications in cell-free DNA (cfDNA), circulating tumor DNA (ctDNA), minimal residual disease (MRD), single-cell analysis, spatial diagnostics, or any combination thereof.

According to one or more embodiments, fluorophore-conjugated oligonucleotide primers are attached to a solid surface of a chip, and samples are hybridized to the chip, allowing for high-throughput detection of a panel of mutations.

### Examples

In a standard intraoperative pathological examination, tissue biopsy was embedded into Optimal cutting temperature compound (OCT) prior to frozen sectioning and mounting onto slides for pathological evaluation. To validate the technology described herein (also referred to subsequently as SNPsnipe technology) with surgical samples, a retrospective pre-clinical correlation study was conducted at Duke University using OCT-embedded brain tissues preserved in liquid nitrogen at The Preston Robert Tisch Brain Tumor Center. One cryo-sectioned scroll at 10-micron thickness from each specimen was lysed in 150 microliters (μl) SNPsnipe Lysis Buffer, and 4 μl lysate supernatant was used for each SNPsnipe assay reaction. All tissue preparation and SNPsnipe assays were performed independently by the Duke Brain Tumor Biorepository and the Duke Molecular Physiology Institute.

Fifteen (15) patient brain tumor OCT specimens with available NGS data were selected, including: 5 glioblastomas (IDH1 wild type, TERT C228T), 5 astrocytomas (IDH1 R132H, TERT wild type), and 5 oligodendrogliomas (IDH1 R132H, TERT C228T). All samples were blind tested with SNPsnipe assays. The total runtime for a SNPsnipe test was about 20 minutes, including 5 minutes of sample lysis and 15 minutes of reaction time. As shown in Table 1, both the IDH1 R132H assay and TERT C228T assays demonstrated 100% (10/10) Positive Percent Agreement (PPA) and 100% (5/5) Negative Percent Agreement (NPA) with the NGS methods. This result provided strong proof of principle for the accuracy and feasibility of the SNPsnipe assay using clinical-relevant samples in a proposed time frame (˜20 minutes from sample to result).

TABLE 1
Comparison of SNPsnipe assays with NGS sequencing
results using clinical frozen specimens

| | NGS method | | |
|---|---|---|---|
| | | Mutation | |
| | Mutation | Not | |
| | Detected | Detected | Total |
| | n | n | n |
| SNPsnipe | | | |
| IDH1 R132H | | | |
| Mutation Detected | 10 | 0 | 10 |
| Mutation Not Detected | 0 | 5 | 5 |
| Total | 10 | 5 | 15 |
| SNPsnipe | | | |
| TERT C228T | | | |
| Mutation Detected | 10 | 0 | 10 |
| Mutation Not Detected | 0 | 5 | 5 |
| Total | 10 | 5 | 15 |

The figures provided herein are not necessarily to scale, although a person skilled in the art will recognize instances where the figures are to scale and/or what a typical size is when the drawings are not to scale. While in some embodiments movement of one component is described with respect to another, a person skilled in the art will recognize that other movements are possible. Additionally, a number of terms may be used throughout the disclosure interchangeably but will be understood by a person skilled in the art. Further, to the extent features, sides, or steps are described as being “first” or “second,” such numerical ordering is generally arbitrary, and thus such numbering can be interchangeable. Still further, in the present disclosure, like-numbered components of various embodiments generally have similar features when those components are of a similar nature and/or serve a similar purpose. Lastly, the present disclosure includes some illustrations and descriptions that include prototypes, bench models, or experimental design. A person skilled in the art will recognize how to rely upon the present disclosure to integrate the techniques, systems, devices, and methods provided for into a product in view of the present disclosures.

While the concepts of the present disclosure are susceptible to various modifications and alternative forms, specific exemplary embodiments of the disclosure have been shown by way of example. It should be understood, however, that there is no intent to limit the concepts of the present disclosure to the particular disclosed forms; the intention is to cover all modifications, equivalents, and alternatives falling within the spirit and scope of the invention as defined by the claims. Although this disclosure refers to specific embodiments, it will be understood by those skilled in the art that various changes in form and detail may be made without departing from the subject matter set forth in the accompanying claims.

1. A system for identifying a single-nucleotide polymorphism (SNP) comprising:
an oligonucleotide primer comprising a SNP recognition sequence;
a fluorophore conjugated to the oligonucleotide primer at a conjugation site to form a fluorophore-conjugated oligonucleotide primer;
a polymerase enzyme to extend the oligonucleotide primer upon binding to an oligonucleotide strand with a complimentary sequence to the oligonucleotide primer, forming a double stranded deoxyribonucleic acid (DNA) amplicon; and
wherein the fluorophore-conjugated oligonucleotide primer is a sole primer in the system, such that the fluorophore only emits a fluorescent signal when the fluorophore-conjugated oligonucleotide primer binds to a sequence specifically complementary to the SNP recognition sequence.

2. The system of claim 1, further comprising an amplification platform.

3. The system of claim 2, wherein the amplification platform is a loop-mediated isothermal amplification (LAMP) platform.

4. The system of claim 1, wherein the fluorophore is in a quenched state when the fluorophore-conjugated oligonucleotide primer does not undergo extension.

5. The system of claim 1, wherein the fluorophore is in a quenched state when a complementary strand formed in the double stranded DNA amplicon contains a mismatch at the conjugation site.

6. The system of claim 1, wherein the fluorophore is in a quenched state when a complementary strand formed in the double stranded DNA amplicon contains a mismatch within two or more bases of the conjugation site.

7. The system of claim 1, wherein the fluorophore-conjugated oligonucleotide primer is a loop primer.

8. The system of claim 1, wherein the fluorophore is conjugated to a guanine (dG), adenine (dA), cytosine (dC), or thymine (dT).

9. The system of claim 8, wherein the fluorophore is conjugated to an oligonucleotide base that is about 1 to about 5 nucleotides from the 3′ end of the oligonucleotide primer.

10. The system of claim 1, wherein the system does not include a reverse primer.

11. A method of treating a patient, the method comprising:
obtaining a sample from the patient, the sample comprising an oligonucleotide;
lysing and preparing the sample for an assay to identify a single-nucleotide polymorphism (SNP);
combining a fluorophore-conjugated oligonucleotide primer and a polymerase enzyme with the oligonucleotide, a fluorophore conjugated to the fluorophore-conjugated oligonucleotide primer at a conjugation site and the fluorophore-conjugated oligonucleotide primer comprising a SNP recognition sequence;
determining whether the oligonucleotide in the sample comprises the SNP based on whether the fluorophore emits a fluorescent signal, the fluorophore-conjugated oligonucleotide primer being a sole primer in the method, such that the fluorophore only emits the fluorescent signal when the fluorophore-conjugated oligonucleotide primer binds to a sequence specifically complementary to the SNP recognition sequence; and
treating the patient if the SNP is detected in the sample.

12. The method of claim 11, wherein determining whether the oligonucleotide in the sample comprises determining if the fluorophore remains quenched, indicating a DNA duplex mismatch.

13. The method of claim 11, wherein treating the patient includes performing a molecular diagnosis for a condition or a disease, wherein the condition or the disease is, optionally, tuberculosis (TB), COVID-19, meningitis, encephalitis, congenital infections, sepsis, an acute coronary syndrome, a histocompatibility issue, an adverse drug reaction, pre-eclampsia, cancer, or any combination thereof.

14. The method of claim 11, further comprising employing a digital platform, wherein optionally, the digital platform is for applications in circulating cell free DNA, circulating cell free RNA, circulating tumor DNA (ctDNA), circulating tumor RNA (ctRNA), circulating pathogen, minimal residual disease (MRD), single-cell analysis, and/or spatial diagnostics.

15. A method for identifying a single-nucleotide polymorphism (SNP) in an oligonucleotide sample, the method comprising:
combining a fluorophore-conjugated oligonucleotide primer comprising a SNP recognition sequence, a polymerase enzyme, and the oligonucleotide sample, a fluorophore conjugated to the fluorophore-conjugated oligonucleotide primer at a conjugation site;
determining whether the oligonucleotide sample comprises the SNP based on whether the fluorophore emits a fluorescent signal; and
confirming that the oligonucleotide sample includes the SNP when the fluorescent signal is emitted;
wherein the fluorophore-conjugated oligonucleotide primer is a sole primer in the method, such that the fluorophore only emits the fluorescent signal when the fluorophore-conjugated oligonucleotide primer binds to a sequence specifically complementary to the SNP recognition sequence.

16. The method of claim 11, wherein the fluorophore-conjugated oligonucleotide primer is a loop primer.

17. The method of claim 11, wherein the fluorophore-conjugated oligonucleotide primer further includes one or more Locked Nucleic Acids (LNAs).

18. The method of claim 15, wherein the method does not include using a reverse primer.
